(19) United States
(12) Patent Application Publication
(10) Pub. No.: US 2024/0307302 A1
(43) Pub. Date: Sep. 19, 2024

(54) NOVEL OMEGA 3 CARRIER PREPARATIONS FOR INHALATION DRUG DELIVERY FOR TREATING LUNG INFLAMMATION

(71) Applicant: LEIUTIS PHARMACEUTICALS LLP, Hyderabad (IN)

(72) Inventors: Chandrashekhar Kocherlakota, Secunderabad (IN); James Thomas Brenna, Austin, TX (US); Sesha Durga Kumar Kothapalli, Round Rock, TX (US); Nagaraju Banda, Hyderabad (IN); Arjun Narala, Warangal (IN); Srinath Akula, Hyderabad (IN)

(21) Appl. No.: 18/681,202

(22) PCT Filed: Aug. 3, 2022
(86) PCT No.: PCT/IB2022/057208

§ 371 Date: Feb. 5, 2024

(30) Foreign Application Priority Data
Aug. 4, 2021 (IN) 202141035170
Nov. 23, 2021 (IN) 202141053853

(51) Int. Cl. A61K 9/00; A61K 31/00; A61K 31/202; A61K 31/4045; A61K 31/58; A61K 47/24

(52) U.S. Cl. CPC A61K 9/0078; A61K 31/202; A61K 31/4045; A61K 31/58; A61K 31/658; A61K 47/24

(57) ABSTRACT

Inflammatory disorders of the lung are induced by COVID-19 disease, asthma, and numerous other disorders. Omega-3 fatty acids (O3FA), particularly docosahexaenoic acid (DHA), docosapentaenoic acid (DPA) or cicosapentaenoic acid (EPA), have known anti-inflammatory and pro-resolving properties. Reduction of lung pathology and inflammation will be accomplished by a mixture incorporating O3FA that is suitable for nebulization using formulas and nebulization apparatus known in the art. The O3FA may be delivered in the form of ethyl esters (EE) or free fatty acid (FEA) oils or non-phosphate-containing glycerolipids or as phospholipids (PL) or as any other form known in the art. The mixture can also be delivered in any of the other forms of inhaled droplet or solids for inhalation known in the art. The patient will inhale for a therapeutically effective period of time to reduce inflammation and improve breathing.

The present invention provides a novel form of pharmaceutical grade Omega 3 fatty acids (O3FA or OFA), specifically docosahexaenoic acid (DHA), docosapentaenoic acid (DPA) and/or eicosapentaenoic acid (EPA) medicament delivery to lungs via nebulization or othermeans for direct inhalation to reduce inflammation associated with medical conditions including COVID-19, asthma and numerous other disorders.

### BACKGROUND OF THE INVENTION

Omega-3 (ω3 or n-3), highly unsaturated fatty acids (HUFA) especially, docosahexaenoic acid (DHA, 22:6n-3), docosapentaenoic acid (DPA, 22:5n-3) and eicosapentaenoic acid (EPA, 20:5n-3) are natural healthy fats found in sea foods like fish, fish oils, squid oils, krill oils, marine oil supplements and microalgae. Plant-derived omega-3s come in the form of alpha-linolenic acid (ALA) which is the only essential omega-3 fatty acid. The body naturally converts ALA into longer chain omega-3 fatty acids, docosahexaenoic acid (DHA), and eicosapentaenoic acid (EPA). Plant sources include nuts and seeds such as chia seeds, Brussels sprouts, walnuts, flax seeds, soybean oil which are rich in ALA and also green leafy vegetables and beans with smaller amounts. Omega-3 are ubiquitous in mammalian tissue, are bioactive components of cell membrane phospholipids, anchor proteins in cell membranes, and serve as precursors to signaling molecules, for instance, eicosanoids and docosanoids. Their modification by biosynthetic inhibition or receptor-mediated actions remains a prevailing strategy for developing valuable drug targets used over-the-counter and prescription drugs such as aspirin, non-steroidal anti-inflammatory drugs (ibuprofen and naproxen) and leukotriene receptor inhibitors (zafirlukast, montelukast, and zileuton). Eicosanoids and docosanoids have wide-ranging functions in the body's cardiovascular, pulmonary, neurological, immune, and endocrine systems.

The global COVID-19 pandemic caused by the SARS-COV-2 virus is an enigma inpart because of its extraordinary range of symptoms, from complete silence that may include person-to-person through-air transmissibility to respiratory failure and death within days of diagnosis. Since the first COVID-19 cases emerged in Wuhan, China, about 220 countries and territories have been affected with over 183,849,133 positive diagnoses and 3,979,872 deaths worldwide as of Jul. 2, 2021. The term “cytokine storm”, previously limited to technical journals, has made its way into the popular press because of the severity of runaway inflammation. An additional defining event is widespread thrombosis, with pathological manifestations in the lung similar to SARS and MERS. Platelet-fibrin thrombi in small arterial vessels are consistent with a coagulopathy. In the end stage, multiple organ failure with severe liver damage is found, consistent with thrombotic microangiopathy. Both inflammation and thrombosis are mediated by signaling molecules derived from HUFA or more precisely the relative mix of them present at any one time in membranes.

Beyond the general inflammatory/prothrombotic potential of the HUFA milieu, the spike protein of the (2003) SARS-COV virus induces cylooxygenase-2 (COX-2), one of the key synthetic enzymes for eicosanoid synthesis. Moreover, induction of COX-2 may be required for efficient early stage replication of mouse hepatitis virus, also a coronavirus. In so far as this is true for SARS-COV-2, individuals with a robust COX-2 response to viral COX-2 induction and thus supportive of the rapid replication of the virus, would be particularly susceptible to a prothrombotic/proinflammatory HUFA milieu. Inhibition of COX-2 via known inhibitors (e.g. celecoxib (Celebrex®)) at the early infection stage would be expected to reduce viral replication. Selective COX-2 inhibitors are effective against arthritis as are high dose omega-3 HUFA, and randomized controlled trial (RCT) evidence indicates regular consumption of omega-3 rich salmon in the context of an “anti-inflammatory diet portfolio” reduces rheumatoid arthritis symptoms. Most selective COX-2 inhibitors were removed from the market because of enhanced thrombotic events, ascribed to rebalancing of the eicosanoid milieu toward thromboxanes and thromboxane A2 production via COX-1. Because severe COVID-19 appears to be an inherently prothrombotic event, selective COX-2 inhibitors may not be effective against it and possibly exacerbate symptoms.

However, a balanced HUFA milieu may be particularly important for avoidance of a COX-2-enhanced cytokine storm or the hypercoagulopathy with features characteristic of a thrombotic storm. Inherited genetic risk factors can enhance or have an additive effect in increasing the risk of thrombotic events during hypercoagulable periods such as severe COVID-19.

Treatment options for lung inflammation may include corticosteroid or glucocorticoid or leukotriene receptor antagonist or many other medications, such as budesonide, prednisone, methylprednisolone, hydrocortisone or montelukast. However, many of these medications come with unwanted side effects that could add additional health risks, or cause physical discomfort. Omega-3 fatty acids (O3FA) are natural, dietary and also available as supplements. Omega-3 fatty acids have been shown to protect against several types of lung diseases such as COVID-19, asthma, cystic fibrosis, COPD, pneumonia, tuberculosis, emphysema, pulmonary edema, lung cancer, acute respiratory distress syndrome (ARDS), asbestosis, bronchiectasis, interstitial lung disease (ILD) including sarcoidosis, idiopathic pulmonary fibrosis, and autoimmune diseases.

Omega-3 fatty acids are always administered systemically primarily orally and less commonly by intravenous infusion. When administered orally they are provided primarily in four common forms: as ethyl esters (EE), as non-phosphate-containing glycerolipids selected from the group comprising triacylglycerol (TG), diacylglycerol, and/or monoacylglycerol, as phospholipids (PL) and as free fatty acids (FFA), also known as non-esterified fatty acids (NEFA). In foods, TAG and PL are the overwhelmingly predominant forms, with small amounts of FFA. Only small amounts of FA, EE are present in humans, usually endogenously synthesized upon consumption of ethanol (alcohol).FA EE usually synthesized by industrial processes from the natural forms, primarily TAG, for f further purification. When administered intravenously, omega-3 are primarily provided as TAG as an emulsion with smaller amounts in PL that may originate with emulsifying agents such as PL. FA EE may be administered IV as emulsions.

In any of these forms, systemic administration results in rapid hydrolysis (“lipolysis”) of all forms, where the resulting liberated FFA enter normal biochemical pathways present to transport and distribute FA to the blood stream to perfuse all organs. Smaller amounts of O3FA are captured and re-esterified into the various lipid classes in cells. In the bloodstream, O3FA are rapidly taken up inan untargeted manner into all tissues thus distributing the oral or intravenous dose to all organs. Thus, only a fraction of any given dose will be incorporated into any particular tissue, such as the lung.

Bioactivity/efficacy of O3FA against pathologies depend on their concentrations intarget tissue and more specifically target lipids of target tissue. For instance, efficacy against lung pathology depends on specific concentration of O3FA in lungtissue and more specifically the concentration in PL present in cell membranes and possibly surfactant lipids. Because of the untargeted nature of systemic administration, any particular dose is less efficacious than an equivalent dose delivered directly to the target organ. More specifically, any particular dose will be less efficacious for treating lung pathology when administered systemically as one of the common forms compared to an equivalent dose administered directly to the lung.

Lung lipids are unique among tissues because of the necessary high secretion of lung surfactant lipids. Lung surfactant is required to lower surface tension and enable a large surface area for gas exchange. Lung surfactant is made up of highly saturated PL.

### BRIEF DESCRIPTION OF THE INVENTION

The present invention provides a novel pharmaceutical composition, which may be administrable in the form of inhalation or nebulization for the reduction of risk factors associated with lung inflammation. The present invention is also directed to a method for treating inflammation-related lung disorders, for example, bronchiolitis, asthma, cystic fibrosis, COPD, pneumonia, tuberculosis, emphysema, pulmonary 30 edema, lung cancer, acute respiratory distress syndrome (ARDS), asbestosis, bronchiectasis, interstitial lung disease (ILD) including sarcoidosis, idiopathic pulmonary fibrosis, and autoimmune diseases, acute pulmonary thrombosis, acute or chronic pulmonary inflammation, inflammatory conditions of the heart and its blood vessels or to alleviate bronchoconstriction optionally in combination with a fast acting bronchodilator and conditions related to acute respiratory distress like COVID-19; and CNS disordes including inflammatory CNS disorders. Patients can be of any age from new born to old age.

The present invention provides a novel pharmaceutical composition of omega 3 fatty acids, comprising therapeutically effective dose of docosahexaenoic acid (DHA), docosapentaenoic acid (DPA), or eicosapentaenoic acid (EPA) or a combination of any two or all the three of DHA, DPA, and EPA. These fatty acids are present as free fatty acids (FFA) or ethyl esters (EE) or phospholipids (PL) or non-phosphate-containing glycerolipids selected from the group comprising triacylglycerol (TG), diacylglycerol, and/or monoacylglycerol.

The pharmaceutical composition may include a pharmaceutically acceptable carrier and O3FA or combinations of O3FA plus melatonin or budesonide. In another embodiment, the pharmaceutical composition comprises O3FA alone, O3FA plus CBD or O3FA plus phospholipids or O3FA plus glycerolipids, combinations of O3FA and other drugs thereof. The pharmaceutical composition comprising the active ingredients may be administered by inhalation or nebulization or other route compatible with inhalation. The compositions of the present invention can be administered to humans or animals to treat lung inflammation. The present composition may be formulated as a suspension or an emulsion and is safe as it includes dietary and natural O3FA.

The present formulation comprises a therapeutically effective dose of phospholipids and/or glycerolipids in which they deliver docosahexaenoic acid (DHA), docosapentaenoic acid (DPA), or eicosapentaenoic acid (EPA) or any combination of two or all three of DHA, DPA, and EPA.

### DETAILED DESCRIPTION OF THE INVENTION

High amounts of pro-inflammatory omega-6 arachidonic acid (AA) suppress anti-inflammatory omega-3 DHA, DPA and EPA synthesis and accumulation in cell membranes. The AA derived eicosanoids regulate immunopathological processes ranging from inflammatory responses to tissue remodeling. AA-derived prostaglandin (PG) synthesis occurs upon release of AA within the membranes by PLA2, thus PG synthesis is limited by the supply of AA. DHA that substitutes for AA is a known inhibitor of PG synthesis by cyclooxygenase. Dietary EPA and AA compete for incorporation into membrane phospholipids but also for biosynthesis from their respective FA precursors, as they share same enzyme for their biosynthesis. AA derived metabolites mediate inflammation in lung disorders, whereas, DHA, DPA and EPA derived metabolites resolve inflammation and clotting.

This invention is a convenient and highly effective method of treating lung inflammation via inhalation routes. The formulations were tested in lipopolysaccharide (LPS) induced acute lung inflammation Wistar rat model. Male rats were chosen for our study and were 6-8 weeks of age at the time of dosing. Ratswere selected and grouped based on animal body weights using stratified randomization method. After randomization, rats were divided into eleven groups having 6 rats in each group (Table 16).

Multiple animal and human studies indicate that both inhaled and systemic corticosteroids cause immunosuppression and impair induction of anti-viral type-I interferon responses to a range of respiratory viruses, including COVID-19. Theseare usually undesirable side effects. In India and other regions of the world, a significant increase in the incidence of fungal infections such as invasive aspergillosis or mucormycosis a life-threatening angioinvasive maxillofacial fungal infection(s) due to corticosteroid administration has been reported in many individuals suffering from COVID-19 especially in patients with diabetes. Inpatients with overwhelming viral illness, broad immunosuppression may be inadvisable. The novel invention represents a significant advantage over corticosteroids. O3FA are dietary and natural, are anti-inflammatory and anti-thrombotic, and safety and efficacy of oral and intravenous lipid emulsions has been established in young children and adult. A double-blind, randomized clinical trial showed O3FA improved the levels of several respiratory and renal function parameters in critically ill COVID-19 patients. The present invention includes administration of O3FA without substantial adverse reactions or side effects.

The present inventors have discovered novel anti-inflammatory preparations using O3FA. In rat experiments the relative content of DHA, DPA and EPA caused significant reduction of lung pathology and inflammation. Inventors also used a combination of O3FA with melatonin or budesonide, O3FA with cannabinoids (CBD), O3FA with phospholipids and glycerolipids which also showed reduction in the inflammation. Other combinations include O3FA with pirfenidone, apremilast, roflumilast, tiotropium bromide, nintedanib, isoniazid, streptomycin, tetrahydrocannabinol (THC), montelukast and other additional active ingredients thereof.

The pharmaceutical composition in the present invention can be used to treat acute symptoms or can be used as a “continuing regimen”. In the continuing regimen the pharmaceutical composition can be administered as required to alleviate symptoms and the dosage of each administration can be the same or varied depending on the symptom's improvement. The present pharmaceutical composition can also be used to correct local O3FA deficiency which can correct local inflammation caused by pro-inflammatory omega-6 fatty acids like arachidonic acid.

The present invention provides a method for the treatment of lung inflammatory disorders such as asthma, chronic obstructive pulmonary disease and chronic sinusitis, including cystic fibrosis, interstitial fibrosis, COVID-19 and other disorders of the lung such as bronchiolitis, pneumonia, tuberculosis, emphysema, pulmonary edema, lung cancer, acute respiratory distress syndrome (ARDS), asbestosis, bronchiectasis, interstitial lung disease (ILD) including sarcoidosis, idiopathic pulmonary fibrosis, autoimmune diseases and CNS disorders. According to the embodiments, the method involves administration, of anti-inflammatory and anti-thrombotic natural O3FA agents via inhalation by nebulization routes. The anti-inflammatory and anti-thrombotic agents can be administered alone or with one or more additives for example melatonin, CBD, phospholipids. The O3FA can be an esterified component of phospholipids.

The present inventors have discovered the first time O3FA formulations delivered via nebulization either as suspension or emulsion that reduce LPS-induced acute lung inflammation. These formulations are administered to patients for whom NSAIDs are contraindicated.

O3FA were delivered primarily in the form of FFA to enhance incorporation into lung tissue and minimize lipoid pneumonia. The single treatment group using ethyl esters (O3EE) was in the most common form of oral 03 supplements. No symptoms related to excess lipid accumulation was observed, and the O3EE treatment was among the most effective in treating effects of LPS.

The long haulers of COVID-19 are person who have survived the acute disease andhave long term symptoms. They were found to suffer from fibromyalgia, fatigue and sleep disturbance. The O3FA, due to their anti-inflammatory effects, are knownto be beneficial in the treatment of arthritis and neuropathic pain associated with fibromyalgia syndrome (FMS). Melatonin has helped in reducing anxiety, lung fibrosis and controlling insomnia in COVID-19 patients. In this embodiment O3FA and melatonin combination can be administered to resolve long hauler FMS and help patients get better sleep.

Omega-3 FA are precursors for the synthesis of endocannabinoids. Omega-3 FA derived endocannabinoid epoxides have powerful anti-inflammatory properties. Cannabidiol exerts a wide range of anti-inflammation and immunomodulation effects and can mitigate the uncontrolled cytokine storm during acute lung injury. In this embodiment the dual administration of O3FA and CBD is used to resolve lung inflammation in COVID-19 patients.

COPD caused 3.23 million deaths in 2019 and is the third leading cause of death worldwide. Elevated IL-6 levels in the exhaled breath condensate samples are associated with airway inflammation in COPD patients. TNFa over-expression in both humans and animal models showed pathological changes consistent with both emphysema and pulmonary fibrosis. Mice lung histology and computed tomography images showed changes involving airspace enlargement, loss of small airspaces, increased collagen and thickened pleural septa. Increased expression of TGF-β is seen in lung specimens collected from COPD patients. IL-10 levels are elevated in COPD patients. Elevated serum IL-1β levels are associated with airway inflammation in COPD patients. Higher intake of omega-3 fatty acids is associated with lower risk of severe exacerbations, better health-related quality of life, and fewer respiratory symptoms in COPD patients.

Several studies have shown IL-6 as the critical tumor-promoting cytokines in NSCLC. IL-6 levels are increased in the serum and exhaled breath condensate samples from NSCLC patients and are related to tumor size. By inducing epithelial-mesenchymal transition of lung cancer cells IL-6 and TNF-α can promote invasion and metastasis in NSCLC. Increased TGF-β expression was found to be associated with lymph node metastasis and tumor angiogenesis in NSCLC. In late stage NSCLC patients increased expression of IL-10 is seen in tumor-associated macrophages. IL-1β is a key mediator of the initiation of inflammatory response in NSCLC and a potent inducer of the COX2-PGE2 pathway, leading to immune suppression. Cachexia is frequently observed in lung cancer; omega-3 oral supplementation preserved body weight in NSCLC patients undergoing chemoradiotherapy. Lung cancer patients whose plasma phospholipid EPA concentrations were higher showed better preservation of body weight. Most of our O3FA test treatments reduced the levels of IL-6, TNF-α, TGF-β, IL-10 and IL-1β significantly.

The following are the examples provided for illustration purpose of the present invention and do not limit its scope.

### EXAMPLES

The following embodiments were tested in animal models

| S. No | Group | Treatment | Diluent/Formulation |
|---|---|---|---|
| 1 | Group 1 | Normal Control | Normal Saline |
| 2 | Group 2 | Disease Control | Normal Saline + LPS |
| 3 | Group 3 | Test Formulation - 1 | Placebo Emulsion |
| 4 | Group 4 | Test Formulation - 2 | OFA Emulsion (50 mg/mL) |
| 5 | Group 5 | Test Formulation - 3 | OFA Emulsion Diluted |
| | | | to 25 mg/mL with |
| | | | Normal Saline |
| 6 | Group 6 | Test Formulation - 4 | FO Emulsion |
| 7 | Group 7 | Reference | Budesonide (0.25 mg/mL) |
| | | Formulation - 1 | |
| 8 | Group 8 | Reference | Montelukast Sodium (4 |
| | | Formulation - 2 | mg/mL) |
| 9 | Group 9 | Reference | Melatonin (1 mg/mL) in |
| | | Formulation - 3 | OFA |
| 10 | Group 10 | — | Montelukast Sodium (4 |
| | | | mg/mL) + OFA Emulsion |
| 11 | Group 11 | Test Formulation - 5 | OFA + LPC Emulsion |

### Example 1: (Test Formulation—1; Group—3)

TABLE 1
Composition of Placebo Emulsion

| | | Qty Per mL | |
|---|---|---|---|
| S. No | Name of the Ingredients | (mg) | % (w/v) |
| 1 | Lipoid E 80 S | 12.5 | 1.25 |
| 2 | Glycerol | 20 | 2.0 |
| 3 | Sodium Hydrogen Carbonate | 2.5 | 0.25 |
| 4 | Milli-Q Water | Q.S to 1 mL | Q.S to 100% |

### Manufacturing Procedure:

All the ingredients were weighed as per manufacturing formula. 90% Milli-Q water was taken in a manufacturing vessel to which glycerol and sodium hydrogen carbonate was added and dissolved by stirring at 400 rpm for 3 minutes. To the above solution, Lipoid E 80 S was added and dissolved by heating on a water bath maintained at a temperature at 55° C. (50° C. to 60° C.).

### Preparation of Emulsion:

High Shear Homogenization: The obtained mixture was homogenized at 8500 rpm by maintaining the product temperature at 45° C. (40° C. to 50° C.) for 15 min. Later, the volume was adjusted up to required level with remaining quantity of milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

TABLE 2
Physical Parameters data of Placebo Emulsion

| S. No | Parameter | Result |
|---|---|---|
| 1 | pH | 9.12 |
| 2 | Osmolality (mOsm/Kg) | 223 |
| 3 | Density (g/cc) | 1.003 |
| 4 | Viscosity (mPas) | 1.075 |

### Example 2: (Test Formulation—2; Group—4)

TABLE 3
Composition of Omega Fatty Acid Emulsion

| | | Qty Per mL | |
|---|---|---|---|
| S. No | Name of the Ingredients | (mg) | % (w/v) |
| 1 | Omega Fatty Acid | 50 | 5.0 |
| 2 | Lipoid E 80 S | 8 | 0.8 |
| 3 | Glycerol | 20 | 2.0 |
| 4 | Sodium Hydrogen Carbonate | 2.5 | 0.25 |
| 5 | Milli-Q Water | Q.S to 1 mL | Q.S to 100% |

### Manufacturing Procedure:

All the ingredients were weighed as per manufacturing formula.

Preparation of Oil Phase: Accurately weighed quantity of omega fatty acid oil was taken in a manufacturing vessel to which Lipoid E 80 S was added and dissolved by heating on a water bath at 55° C. (50° C. to 60° C.) to give an oil phase.

Preparation of Aqueous Phase: 90% Milli-Q water was taken in another manufacturing vessel and the aqueous phase was prepared by dissolving glycerol and sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C.(40° C. to 50° C.) for 15 min. Later, the volume was adjusted up to required level with remaining quantity of milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

Physical parameters, particle size distribution and zeta potential (ZP) of the emulsion were determined.

TABLE 4
Particle Size data of Omega Fatty acid Emulsion

| HDD | PDI | D10 | D50 | D90 | | ZP |
|---|---|---|---|---|---|---|
| (nm) | (%) | (nm) | (nm) | (nm) | SPAN | (mV) |
| 145.18 | 21.53 | 80.94 | 143.00 | 233.67 | 1.14 | −75.60 |

TABLE 5
Physical Parameters data of Omega Fatty acid Emulsion

| S. No | Parameter | Result |
|---|---|---|
| 1 | pH | 7.68 |
| 2 | Osmolality (mOsm/Kg) | 255 |
| 3 | Density (g/cc) | 1.001 |
| 4 | Viscosity (mPas) | 1.262 |

### Example 3: (Test Formulation—3; Group—5)-Test formulation—2 Diluted to50 % with Normal Saline

Physical parameters, particle size distribution and zeta potential (ZP) of the emulsion were determined.

TABLE 6
Physical Parameters of OFA Emulsion diluted to 50% with Normal Saline

| HDD | PDI | D10 | D50 | D90 | | ZP | | Osmolality |
|---|---|---|---|---|---|---|---|---|
| (nm) | (%) | (nm) | (nm) | (nm) | SPAN | (mV) | pH | (mOsm/Kg) |
| 141.88 | 18.10 | 83.09 | 134.79 | 210.96 | 0.94 | −79.1 | 7.78 | 121 |

### Example 4: (Test Formulation—4; Group—6)

TABLE 7
Composition of Fish Oil Emulsion

| | | Qty Per mL | |
|---|---|---|---|
| S. No | Name of the Ingredients | (mg) | % (w/v) |
| 1 | Fish Oil (Incromega E3322) | 50 | 5 |
| 2 | Lipoid E 80 S | 12.5 | 1.25 |
| 3 | Glycerol | 20 | 2 |
| 4 | Sodium Hydrogen Carbonate | 0.15 | 0.015 |
| 5 | Milli-Q Water | Q.S to 1 mL | Q.S to 100% |

### Manufacturing Procedure:

All the ingredients were weighed as per manufacturing formula.

Preparation of Oil Phase: Accurately weighed quantity of fish oil was taken in a manufacturing vessel to which Lipoid E 80 S was added and dissolved by heating on a water bath at 55° C. (50° C. to 60° C.) to give an oil phase.

Preparation of Aqueous Phase: 90% Milli-Q water was taken in another manufacturing vessel and the aqueous phase was prepared by dissolving glycerol and sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C.(40° C. to 50° C.) for 15 min. Later, the volume was adjusted up to required level with remaining quantity of milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

TABLE 8
Particle Size data of Fish Oil Emulsion

| HDD | PDI | D10 | D50 | D90 | | ZP |
|---|---|---|---|---|---|---|
| (nm) | (%) | (nm) | (nm) | (nm) | SPAN | (mV) |
| 132.95 | 18.50 | 79.89 | 125.98 | 192.76 | 0.93 | −24.07 |

TABLE 9
Physical Parameters data of Fish Oil Emulsion

| S. No | Parameter | Result |
|---|---|---|
| 1 | pH | 7.43 |
| 2 | Osmolality (mOsm/Kg) | 249 |
| 3 | Density (g/cc) | 0.999 |
| 4 | Viscosity (mPas) | 1.311 |

### Example 5: (Reference Formulation—2; Group—8)

TABLE 10
Composition of Montelukast Sodium Solution

| | Name of the | Qty Per mL | |
|---|---|---|---|
| S. No | Ingredients | (mg) | % (w/v) |
| 1 | Montelukast Sodium | 4 | 0.4 |
| 2 | Milli-Q Water | Q.S to 1 mL | Q.S to 100% |

### Manufacturing Procedure:

90% of Milli-Q water was taken into a manufacturing vessel. Weighed amount of Montelukast Sodium was added and dissolved by stirring at 400 rpm. The volume was adjusted with remaining amount of Milli-Q water. The sample was stored at 2-8° C. and protected from light.

TABLE 11
Physical Parameters data of Montelukast Sodium Solution

| S. No | Parameter | Result |
|---|---|---|
| 1 | pH | 9.13 |
| 2 | Osmolality (mOsm/Kg) | 132 |
| 3 | Density (g/cc) | 1.0 |
| 4 | Viscosity (mPas) | 0.992 |

### Example 6: (Reference Formulation-3; Group-9)

TABLE 12
Composition of Melatonin Solution

| | Name of the | Qty Per mL | |
|---|---|---|---|
| S. No | Ingredients | (mg) | % (w/v) |
| 1 | Melatonin | 1 | 0.1 |
| 2 | Omega Fatty Acid | Q.S to 1 mL | Q.S to 100% |

### Manufacturing Procedure:

90% of omega fatty acid oil was taken into a manufacturing vessel. Weighed amount of Melatonin was added and dissolved by stirring at 400 rpm. The volume was adjusted with remaining amount of OFA. The sample was stored at 2-8° C. and protected from light.

### Example 7: (Test Formulation—5; Group—11)

TABLE 13
Composition of Omega Fatty Acid and LPC Emulsion

| | Name of the | Qty Per mL | |
|---|---|---|---|
| S. No | Ingredients | (mg) | % (w/v) |
| 1 | Omega Fatty Acid | 50 | 5 |
| 2 | Cannabidiol Synthetic (LPC) | 8 | 0.8 |
| 3 | Lipoid E 80 S | 8 | 0.8 |
| 4 | Glycerol | 20 | 2 |
| 5 | Sodium Hydrogen Carbonate | 2.5 | 0.25 |
| 6 | Milli-Q Water | Q.S to 1 mL | Q.S to 100% |

Note:
LPC—Leiutis Proprietary Compound

### Manufacturing Procedure:

All the ingredients were weighed as per manufacturing formula.

Preparation of Oil Phase: Accurately weighed quantity of omega fatty acid oil wastaken in a manufacturing vessel to which egg lecithin was added and dissolved by heating on a water bath at 55° C. (50° C. to 60° C.). The mixture was cooled down to 40° C. and Cannabidiol (LPC) was added and dissolved to give an oil phase.

Preparation of Aqueous Phase: 90% Milli-Q water was taken in another manufacturing vessel and the aqueous phase was prepared by dissolving glycerol and sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C.(40° C. to 50° C.) for 15 min. Later, the volume was adjusted up to required level with remaining quantity of milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000psi, Pass 2 at 18,000 psi and Pass 3 at 18,000 psi followed by subsequent cooling the product to room temperature to obtain emulsion.

Physical parameters, particle size distribution and zeta potential (ZP) of the emulsion were determined.

TABLE 14
Particle Size data of Omega Fatty Acid and LPC Emulsion

| HDD | PDI | D10 | D50 | D90 | | ZP |
|---|---|---|---|---|---|---|
| (nm) | (%) | (nm) | (nm) | (nm) | SPAN | (mV) |
| 157.97 | 22.50 | 78.15 | 161.81 | 302.13 | 1.36 | −62.80 |

TABLE 15
Physical Parameters data of Omega Fatty Acid and LPC Emulsion

| S. No | Parameters | Result |
|---|---|---|
| 1 | pH | 7.87 |
| 2 | Osmolality (mOsm/Kg) | 234 |
| 3 | Density (g/cc) | 1.0 |
| 4 | Viscosity (mPas) | 1.582 |

Acute Lung Inflammation Study. Evaluation of the Efficacy of Test formulations in LPS-induced Acute Lung Inflammation in Wistar Rats Model.

A total of 66 male Wistar rats were taken and assigned into 11 groups (Group 1 (G1) to G11) containing six (06) animals per group (Group (G) description and treatment are presented in Table 16). Technicians performing the experiments were blinded to the treatment/reference and knew only group numbers (G1, G2, etc).

On each day of dosing, a fresh solution of 2 mg/mL of LPS was prepared using sterile normal saline. The animals from group G2 to G11 were dosedintraperitoneally with LPS once per day at the dose volume of 2 mg/kg of body weight. Animals of group G1 did not receive the LPS treatment. LPS was administered once per day; the Test or Reference Item was administered twice per day.

LPS injection was made once a day in the morning. One hour later, the Test or Reference Item treatment were given to the respective groups. The Test or Reference Item treatment was administered again in the afternoon. This continued daily for 7 or 14 days.

All animals were restrained and exposed to Test or Reference item through inhalation using a standard nebulizer. Animals were observed for overt clinical signs during entire period of dosing and till termination of study, and also observed for local effects during and after administration of test item.

All animals showed increase in body weight during experiment period. No mortality was observed during the study period.

Upon arrival at the laboratory, rats were acclimatized to their cages for a period of 7 days with no treatments. None of the animals showed any clinical signs during the acclimatization period. During the treatment period, animals from the groups G6 (Treatment 4), G9 (Treatment 7) and G11 (Treatment 9) showed some clinical signs. Eye irritation and lacrimation were observed immediate after nebulization treatment. These clinical effects subsided within 30 min after nebulization. In the treatment apparatus, rat eyes are exposed to the nebulized Test/Reference vapors.

After 7 days of treatment, were complete, a day later, i.e. day 8 after commencing treatment, 3 rats from each group were anesthetized using isoflurane and blood was collected. Plasma was separated for further analysis. Animals were then euthanized and bronchoalveolar lavage fluid (BALF) and lung tissue samples were collected. Lung tissue samples were quickly harvested, stored in 10%neutral buffered formalin (NBF) for further processing of histopathological analysis.

After 14 days of treatment, were complete, a day later, i.e. day 15 after commencing treatment, the remaining 3 rats from each group were anesthetized using isoflurane and blood was collected. Plasma was separated for further analysis. Animals were then euthanized and bronchoalveolar lavage fluid (BALF) and lung tissue samples were collected. Lung tissue samples were quickly harvested, stored in 10% neutral buffered formalin (NBF) for further processing of histopathological analysis.

### Lung Weights

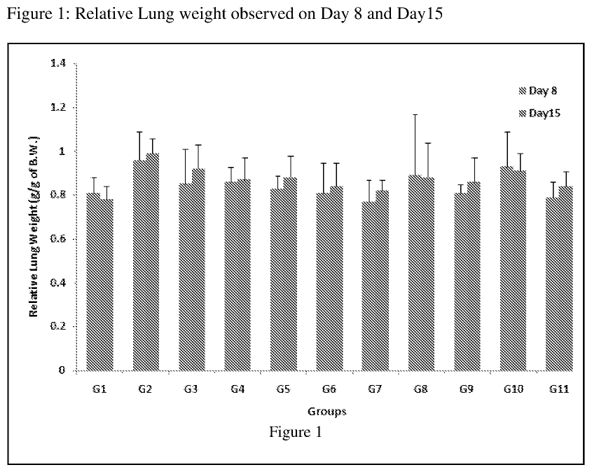

Results are represented as Mean±SD of three rats/time point (day 8 and day 15) in FIG. 1. Relative lung weights of control group (G1) compared with all the treatment groups (G2-G11) using One-way ANOVA followed by Dunnett's multiple comparison. * p<0.05; ** p<0.01; *** p<0.001.

### Lung Histology

Lung tissue samples fixed in 10% NBF were proceeded for histopathology observations. For each animal one slide was prepared and stained with H&E stain.All gross lesions were examined.

### Histopathology Evaluations External Findings—Gross Pathology

External examination of male animals from normal control (G1), disease control (G2) and treatment group (G3, G4, G5, G6, G7, G8, G9, G10 & G11) did not revealany lesions of pathological significance.

### Internal Findings—Gross Pathology

Internal examination of male animals belonging to the normal control (G1), diseasecontrol (G2) and treatment group (G3, G4, G5, G6, G7, G8, G9, G10 & G11) did not reveal any pathological abnormalities.

### Microscopic Findings:

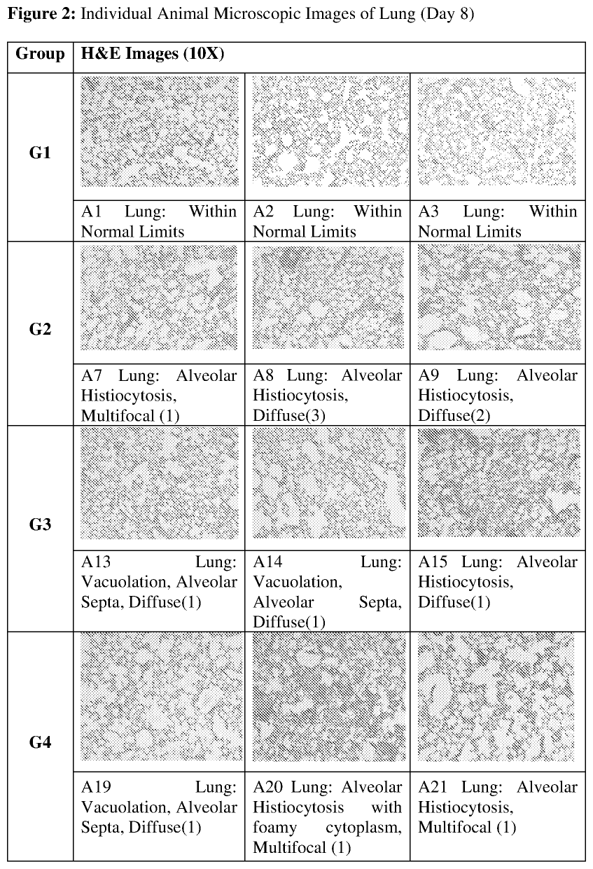

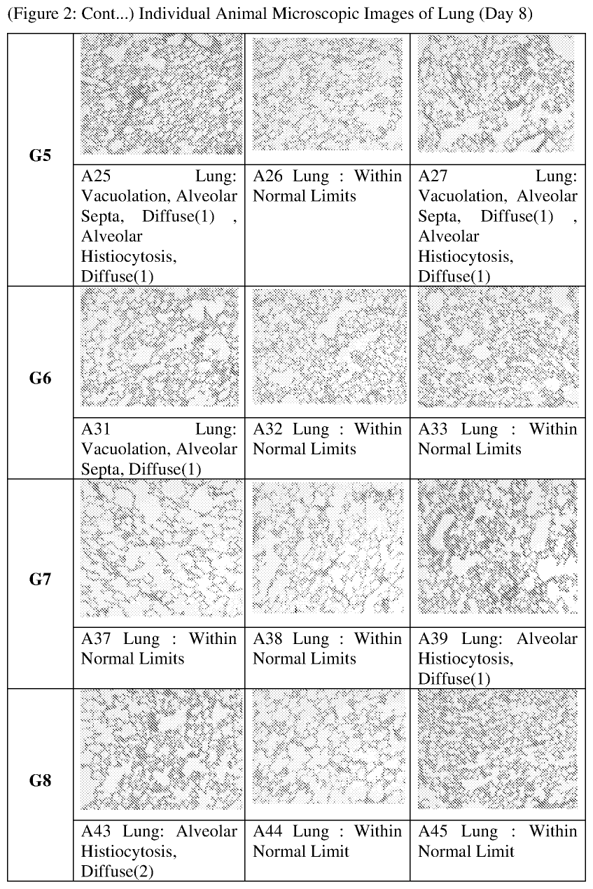

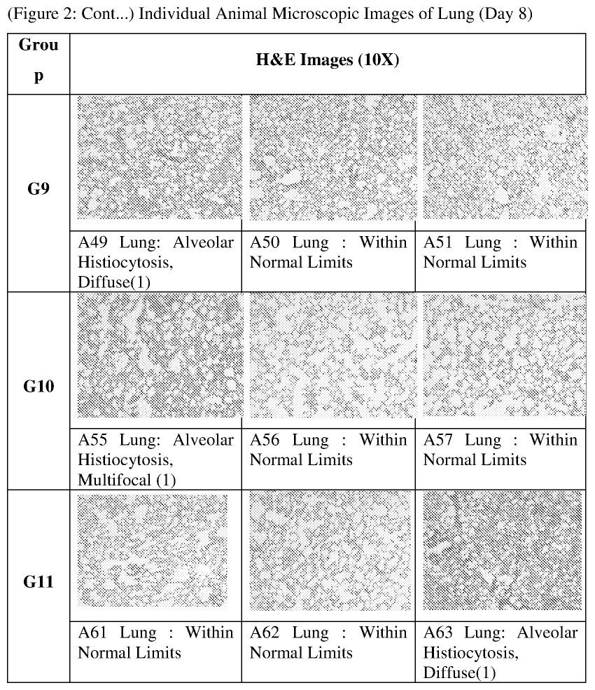

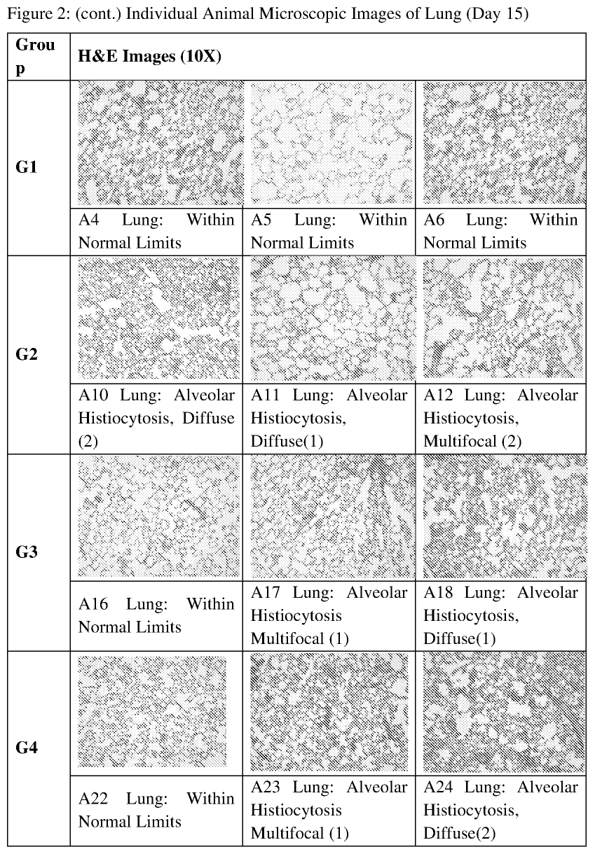

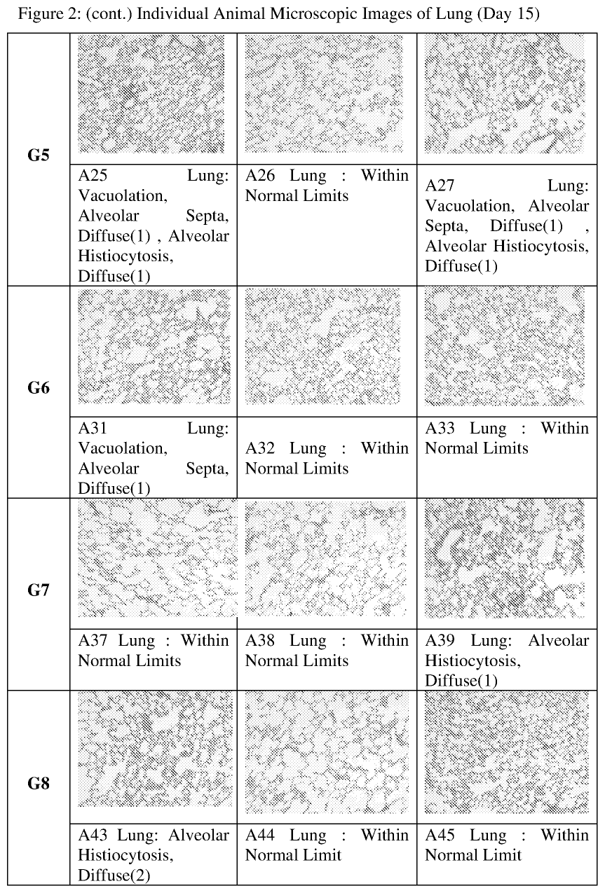

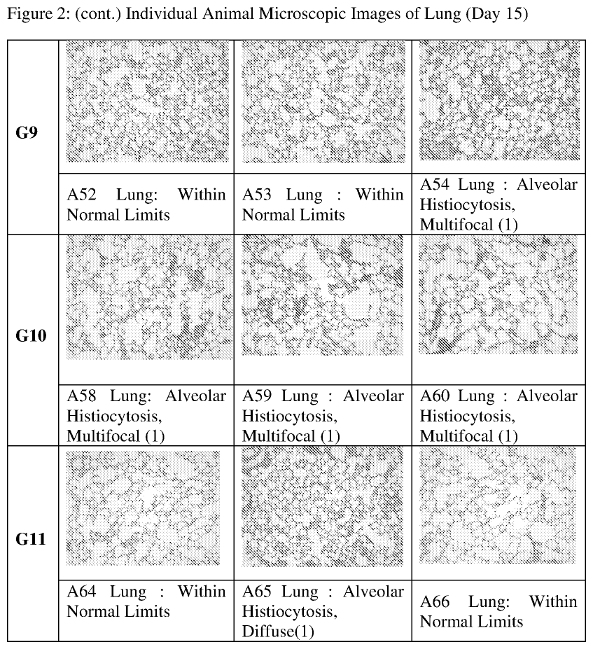

Various microscopic changes in normal control (G1), disease control (G2) and treatment group (G3 to G11) are presented in FIG. 2.

Minimal alveolar histiocytosis [2/6 G2, 3/6 G3, 3/6 G4, 3/6 G5, 1/6 G6, 1/6 G7, 2/6G9, 4/6 G10, & 2/6 G11], slight alveolar Histiocytosis [3/6 G2, 1/6 G4, 1/6 G5, 1/6 G6, & 3/6 G8] and moderate alveolar histiocytosis [1/6 G2] were observed in lung.

Minimal vacuolation, alveolar septum [2/6 G3, 1/6 G4 and 2/6 G5] and slight vacuolation, alveolar septum [1/6 G6] were noted in lung.

Mean severity score for the alveolar histiocytosis in different groups were as follows-viz., G1-0.0, G2-1.83, G3-0.50, G4-0.83, G5-0.83, G6-0.50, G7-0.17, G8-1.0, G9-0.33, G10-0.67 and G11-0.33.

Mean severity score for the vacuolation, alveolar septum in different groups were as follows-viz., G1-0.0, G2-0.0, G3-0.33, G4-0.17, G5-0.33, G6-0.33, G7-0.0, G8-0.0, G9-0.0, G10-0.0 and G11-0.0.

In all results, statistical significance is indicated as follows. * p<0.05; ** p<0.01 ;*** p<0.001.

Budesonide (B-ref) animals had the lowest score, based on one animal scored as slight and the rest normal. The treatments containing O3 scored less than half severity compared to Cd.

Minimal vacuolation was observed in 1-2 animals in the EPL, 03, 03-0.5, and O3EE groups.

### Immune Parameters

The collected BALF samples were analyzed for the IL-6, IL-1β and TNF-α level for all animals. IL-10 and TGF-β were estimated in the plasma samples.

### IL-6

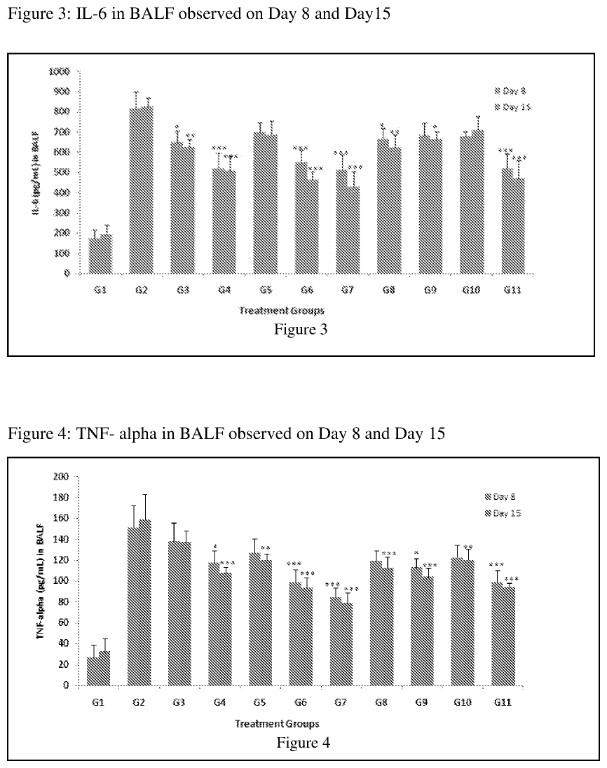

Results of IL-6 levels are presented in FIG. 3 and are represented as Mean +SD of three rats/time point (day 8 and day 15). IL-6 value of LPS control group (G2) compared with all the treatment groups (G3-G11) using One-way ANOVA followed by Dunnett's multiple comparison. * p<0.05; ** p<0.01; *** p<0.001.

### TNF-α

Results of TNF-α levels are presented in FIG. 4 and are represented as Mean±SD of three rats/time point (day 8 and day 15). TNF-α value of LPS control group(G2) compared with all the treatment groups (G3-G11) using One-way ANOVA followed by Dunnett's multiple comparison. * p<0.05; ** p<0.01; *** p<0.001.

### p IL-10

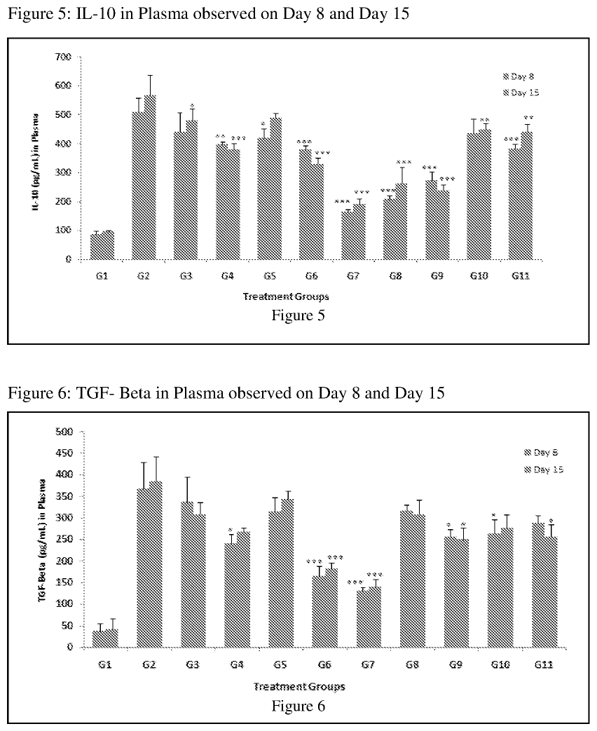

Results of IL-10 levels are presented in FIG. 5 and are represented as Mean ±SDof three rats/time point (day 8 and day 15). IL-10 value of LPS control group (G2)compared with all the treatment groups (G3-G11) using One-way ANOVA followed by Dunnett's multiple comparison. * p<0.05; ** p<0.01; *** p<0.001.

### TGF-β

Results of TGF-β levels are presented in FIG. 6 and are represented as Mean±SD of three rats/time point (day 8 and day 15). TGF-β value of LPS control group (G2) compared with all the treatment groups (G3-G11) using One-way ANOVA followed by Dunnett's multiple comparison. * p<0.05; *** p<0.001

### Interpretation of Immune Data

COVID-19 is an enigma. Multiple studies have shown elevation of both pro-inflammatory and anti-inflammatory cytokines in COVID-19 patients, reviewed by Dhar et al. IL-6 and IL-10 are found to be predictive of COVID-19 disease severity.A dramatic elevation of IL-6 and IL-10 levels is a characteristic feature of the cytokine storm in COVID-19 patients. Persistent viral stimulation, and IL-6, IL-10and TNF-alpha levels, are indicators of T-cell exhaustion in COVID-19 patients. Increased IL-6 and TNF-α levels are significant predictors of COVID-19 severity and death.

In COVID-19 patients, increased pro-inflammatory IL-6 levels are associated with increased body temperature, elevation in CRP and ferritin inflammation markers, pulmonary inflammation and extensive lung damage. The Test treatments reduced the levels of IL-6 significantly (FIG. 3).

Tissue necrosis factor-α (TNF-α) is a well-known pro-inflammatory molecule. It is upregulated in most inflammatory conditions and contributes to changes in blood coagulation. Increased TNF-α along with IL-6 and IL-10 are indicators of a hyper inflammatory response and an underlying cytokine storm in COVID-19patients. An excessive amount of ferritin in COVID-19 patients is also reflective ofa surplus of TNF-α levels. The Test treatments reduced the levels of TNF-a significantly (FIG. 4).

IL-10 is a pleiotropic cytokine whose primary function in most tissues is to limit the inflammatory response, however, in COVID-19 it is dramatically elevated. Thisphenomenon in COVID-19 is thought to be a negative feedback mechanism to suppress inflammation. IL-10 is also known to introduce T-cell anergy during viral infection. The Test treatments reduced the levels of IL-10 significantly compared to the positive control LPS no treatment group (FIG. 5).

Transforming growth factor β (TGF-β) is a pleiotropic cytokine which plays a majorrole in inflammatory conditions. TGF-β along with IL-6 drives the differentiation of T helper 17 (Th17) cells, which promote inflammation and augment autoimmuneconditions. In addition, TGF-β promotes the differentiation of IL-10 producing T cells, which lack suppressive function and in turn promote tissue inflammation. TGF-β promote lung fibrosis in COVID-19 patient. The Test treatments reduced the levels of TGF-β (FIG. 6).

IL-1β is a pro-inflammatory cytokine that is crucial for host-defense responses to infection, antimicrobial immunity and autoimmune inflammation. IL-1β levels are associated with cytokine storm in a subset of COVID-19 patients. IL-1β expression levels are found to be significantly increased in the bronchial wall of asthmatic patients. On the other hand, fish oil treatment decreased IL-18 production in healthy human volunteers. Our O3FA test treatment O3EE reduced the levels of IL-18 significantly at both time points and the reduction was better than the B-Ref group.

TABLE 16
Grouping and Treatment with Test and Reference Item.
After randomization, rats were divided into 11 groups having
6 rats in each group. Animals from each group received the
respective treatment twice daily for 7 days (3 animals/group)
or 14 days (3 animals/group) through nebulization. The
concentration and the dose volume for nebulization for
each group are given in below table:

| | Formulation/ | Concentration | Volume to | Volume used | Nebulization |
|---|---|---|---|---|---|
| Group | Composition | of Solution | be Nebulized | for Nebulization | duration |
| G1 | Normal | NA | 1 mL | 1.5 mL | 2.5 min |
| | Control | | | | |
| | (Normal | | | | |
| | saline | | | | |
| | injection no | | | | |
| | LPS) | | | | |
| G2 | Disease | NA | 1 mL | 1.5 mL | 2.5 min |
| | Control | | | | |
| | (Normal | | | | |
| | saline | | | | |
| | injection) + | | | | |
| | LPS, once | | | | |
| | daily | | | | |
| G3 | Treatment 1 | NA | 1 mL | 1.5 mL | 3.0 min |
| | (Egg | | | | |
| | Lecithin | | | | |
| | Dispersion, | | | | |
| | twice daily) + | | | | |
| | LPS, once | | | | |
| | daily | | | | |
| G4 | Treatment 2 | 50 mg/mL | 1 mL | 1.5 mL | 4.5 min |
| | (OFA | | | | |
| | Dispersion, | | | | |
| | twice daily) + | | | | |
| | LPS, once | | | | |
| | daily | | | | |
| G5 | Treatment 3 | 25 mg/mL | 2 mL | 3.0 mL | 6.0 min |
| | (OFA | | | (1.5 L + | |
| | Dispersion | | | 1.5 mL) | |
| | diluted to | | | | |
| | 50% in NS, | | | | |
| | twice | | | | |
| | daily) + | | | | |
| | LPS, once | | | | |
| | daily | | | | |
| G6 | Treatment 4 | 50 mg/mL | 1 mL | 1.5 mL | 3.0 min |
| | (Fish Oil | | | | |
| | Dispersion, | | | | |
| | twice daily) + | | | | |
| | LPS, once | | | | |
| | daily | | | | |
| G7 | Treatment 5 | 0.5 mg/mL | 1 mL | 1.5 ml | 3.0 min |
| | (Reference: | | | | |
| | Budesonide | | | | |
| | Respules, | | | | |
| | twice daily) + | | | | |
| | LPS, once | | | | |
| | daily | | | | |
| G8 | Treatment 6 | 4 mg/mL | 1 mL | 1.5 ml | 3.0 min |
| | (Reference: | | | | |
| | Montelukast | | | | |
| | sodium | | | | |
| | solution, | | | | |
| | twice daily) + | | | | |
| | LPS, | | | | |
| | once daily | | | | |
| G9 | Treatment 7 | 1.0 mg/mL + | 1 mL | 1.5 ml | 4.5 min |
| | (Melatonin + | 50 mg/mL | | | |
| | OFA | | | | |
| | dispersion, | | | | |
| | twice daily) + | | | | |
| | LPS, once | | | | |
| | daily | | | | |
| G10 | Treatment 8 | 4 mg/mL + | 2 mL | 3.0 mL | 6.5 min |
| | (Reference: | 50 mg/mL | | (1.5 ml + | |
| | Montelukast | | | 1.5 ml) | |
| | sodium | | | | |
| | solution + | | | | |
| | OFA | | | | |
| | Dispersion, | | | | |
| | twice daily) + | | | | |
| | LPS, once | | | | |
| | daily | | | | |
| G11 | Treatment 9 | | | | |
| | (Cannabidiol + | | | | |
| | OFA | | | | |
| | dispersion, | 8 mg/mL + | 1 mL | 1.5 ml | 4 min |
| | twice daily) + | 50 mg/mL | | | |
| | LPS, once | | | | |
| | daily | | | | |

### Example 8

TABLE 17
Composition of OFA EE Emulsion

| | | Qty Per mL | |
|---|---|---|---|
| S. No | Ingredients | (mg) | % (w/v) |
| 1 | Omega-3-Acid Ethyl Ester (OFA EE) | 60 | 6.0 |
| 2 | Egg Lecithin (Lipoid E 80) | 10 | 1.0 |
| 3 | Glycerol | 20 | 2.0 |
| 4 | Sodium Hydrogen Carbonate | 0.1 | 0.01 |
| 5 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.) to give an oil phase.

Preparation of Aqueous Phase: 90% Milli-Q water was taken in another manufacturing vessel and the aqueous phase was prepared by dissolving glycerol by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

Physical parameters, particle size distribution and zeta potential (ZP) of the emulsion were determined.

TABLE 18
Particle Size data of OFA EE Emulsion

| HDD | PDI | D10 | D50 | D90 | | ZP |
|---|---|---|---|---|---|---|
| (nm) | (%) | (nm) | (nm) | (nm) | SPAN | (mV) |
| 190.23 | 23.23 | 106.78 | 184.37 | 292.07 | 1.00 | −23.13 |

TABLE 19
Physical Parameters data of OFA EE Emulsion

| S. No | Parameter | Result |
|---|---|---|
| 1 | pH | 7.50 |
| 2 | Osmolality (m Osm/Kg) | 235 |

### Example 9

TABLE 20
Composition of OFA EE and Cannabidiol (0.5 mg/mL) Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 0.5 | 0.05 |
| 4 | Glycerol | 20 | 2.0 |
| 5 | Sodium Hydrogen Carbonate | 0.1 | 0.01 |
| 6 | Milli-Q Water | Q.S to 1 mL | Q.S to 100% |

### Manufacturing Procedure:

All the ingredients were weighed as per the manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

Physical parameters, particle size distribution and zeta potential (ZP) of the emulsion were determined.

TABLE 21
Particle Size data of OFA EE and
Cannabidiol (0.5 mg/mL) Emulsion

| HDD | PDI | D10 | D50 | D90 | | ZP |
|---|---|---|---|---|---|---|
| (nm) | (%) | (nm) | (nm) | (nm) | SPAN | (mV) |
| 146.50 | 22.00 | 80.18 | 143.50 | 240.40 | 1.01 | −20.90 |

TABLE 22
Physical Parameters data of OFA EE and
Cannabidiol (0.5 mg/mL) Emulsion

| S. No | Parameter | Result |
|---|---|---|
| 1 | pH | 7.60 |
| 2 | Osmolality (m Osm/Kg) | 252 |

### Example 10

TABLE 23
Composition of OFA EE and Cannabidiol (2 mg/mL) Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 2 | 0.2 |
| 4 | Glycerol | 20 | 2.0 |
| 5 | Sodium Hydrogen Carbonate | 0.1 | 0.01 |
| 6 | Milli-Q Water | Q.S to 1 mL | Q.S to 100% |

Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

Physical parameters, particle size distribution and zeta potential (ZP) of the emulsion were determined.

TABLE 24
Particle Size data of OFA EE and
Cannabidiol (2 mg/mL) Emulsion

| HDD | PDI | D10 | D50 | D90 | | ZP |
|---|---|---|---|---|---|---|
| (nm) | (%) | (nm) | (nm) | (nm) | SPAN | (mV) |
| 155.72 | 19.47 | 88.29 | 150.56 | 246.50 | 1.00 | −28.23 |

TABLE 25
Physical Parameters data of OFA EE
and Cannabidiol (2 mg/mL) Emulsion

| S. No | Parameter | Result |
|---|---|---|
| 1 | pH | 7.41 |
| 2 | Osmolality (m Osm/Kg) | 260 |

### Example 11

TABLE 26
Composition of OFA EE and Cannabidiol (4 mg/mL) Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 4 | 0.4 |
| 4 | Glycerol | 20 | 2.0 |
| 5 | Sodium Hydrogen Carbonate | 0.1 | 0.01 |
| 6 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

Physical parameters, particle size distribution and zeta potential (ZP) of the emulsion were determined.

TABLE 27
Particle Size data of OFA EE and
Cannabidiol (4 mg/mL) Emulsion

| HDD | PDI | D10 | D50 | D90 | | ZP |
|---|---|---|---|---|---|---|
| (nm) | (%) | (nm) | (nm) | (nm) | SPAN | (mV) |
| 147.83 | 22.03 | 83.36 | 148.40 | 237.90 | 0.99 | −21.57 |

TABLE 28
Physical Parameters data of OFA EE
and Cannabidiol (4 mg/mL) Emulsion

| S. No | Parameter | Result |
|---|---|---|
| 1 | pH | 7.50 |
| 2 | Osmolality (mOsm/Kg) | 220 |

### Example 12

TABLE 29
Composition of OFA EE and Cannabidiol (8 mg/mL) Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 8 | 0.8 |
| 4 | Glycerol | 20 | 2.0 |
| 5 | Sodium Hydrogen Carbonate | 0.1 | 0.01 |
| 6 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of

Cannabidiol was added to the above mixture and dissolved by heating on a water bath at 45° C. (40° C. to 50° C.) to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

Physical parameters, particle size distribution and zeta potential (ZP) of the emulsion were determined.

TABLE 30
Particle Size data of OFA EE and
Cannabidiol (8 mg/mL) Emulsion

| HDD | PDI | D10 | D50 | D90 | | ZP |
|---|---|---|---|---|---|---|
| (nm) | (%) | (nm) | (nm) | (nm) | SPAN | (mV) |
| 175.30 | 22.40 | 100.03 | 174.12 | 278.90 | 1.07 | −21.23 |

TABLE 31
Physical Parameters data of OFA EE
and Cannabidiol (8 mg/mL) Emulsion

| S. No | Parameter | Result |
|---|---|---|
| 1 | pH | 7.63 |
| 2 | Osmolality (m Osm/Kg) | 236 |

### Example 13

TABLE 32
Composition of OFA EE and Cannabidiol (15 mg/mL) Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 15 | 1.5 |
| 4 | Glycerol | 20 | 2.0 |
| 5 | Sodium Hydrogen Carbonate | 0.1 | 0.01 |
| 6 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

Physical parameters, particle size distribution and zeta potential (ZP) of the emulsion were determined.

TABLE 33
Particle Size data of OFA EE and
Cannabidiol (15 mg/mL) Emulsion

| HDD | PDI | D10 | D50 | D90 | | ZP |
|---|---|---|---|---|---|---|
| (nm) | (%) | (nm) | (nm) | (nm) | SPAN | (mV) |
| 147.59 | 25.20 | 73.93 | 152.60 | 267.20 | 1.26 | −15.73 |

TABLE 34
Physical Parameters data of OFA EE
and Cannabidiol (15 mg/mL) Emulsion

| S. No | Parameter | Result |
|---|---|---|
| 1 | pH | 7.64 |
| 2 | Osmolality (m Osm/Kg) | 256 |

### Example 14

TABLE 35
Composition of OFA EE, Cannabidiol
(8 mg/mL) and Pirfenidone Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 8 | 0.8 |
| 4 | Pirfenidone | 50 | 5.0 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) to give an oil phase.

Preparation of Aqueous Phase: 90% Milli-Q water was taken in another manufacturing vessel and the aqueous phase was prepared by dissolving glycerol and sodium hydrogen carbonate followed by dispersing Pirfenidone by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

### Example 15

TABLE 36
Composition of OFA EE, Cannabidiol
(8 mg/mL) and Apremilast Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 8 | 0.8 |
| 4 | Apremilast | 15 | 1.5 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) followed by dissolving/dispersing Apremilast to give an oil phase.

Preparation of Aqueous Phase: 90% Milli-Q water was taken in another manufacturing vessel and the aqueous phase was prepared by dissolving glycerol and sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

### Example 16

TABLE 37
Composition of OFA EE, Cannabidiol
(8 mg/mL) and Roflumilast Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 8 | 0.8 |
| 4 | Roflumilast | 0.5 | 0.05 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of

Cannabidiol was added to the above mixture and dissolved by heating on a water bath at 45° C. (40° C. to 50° C.) followed by dissolving/dispersing Roflumilast to give an oil phase.

Preparation of Aqueous Phase: 90% Milli-Q water was taken in another manufacturing vessel and the aqueous phase was prepared by dissolving glycerol and sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

### Example 17

TABLE 38
Composition of OFA EE, Cannabidiol
(8 mg/mL) and Tiotropium Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 8 | 0.8 |
| 4 | Tiotropium Bromide | 0.005 | 0.0005 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) to give an oil phase.

Preparation of Aqueous Phase: 90% Milli-Q water was taken in another manufacturing vessel and the aqueous phase was prepared by dissolving glycerol and sodium hydrogen carbonate followed by Tiotropium Bromide by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

### Example 18

TABLE 39
Composition of OFA EE, Cannabidiol
(8 mg/mL) and Nintedanib Emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Cannabidiol | 8 | 0.8 |
| 4 | Nintedanib | 50 | 5.0 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) followed by dissolving/dispersing Nintedanib to give an oil phase.

Preparation of Aqueous Phase: 90% Milli-Q water was taken in another manufacturing vessel and the aqueous phase was prepared by dissolving glycerol and sodium hydrogen carbonate by stirring at 400 rpm on a magnetic stirrer maintaining temperature at 55° C. (50° C. to 60° C.).

### Preparation of Emulsion:

High Shear Homogenization: The oil phase was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 3 passes at different pressures i.e. Pass 1 at 10,000 psi, Pass 2 at 18,000 psi and Pass 3 at 18,000 psi followed by subsequent cooling the product to room temperature to obtain emulsion.

### Example 19

TABLE 40
Composition of OFA EE, Cannabidiol and Streptomycin emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Streptomycin | 50 | 5.0 |
| 4 | Cannabidiol | 4 | 0.4 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) followed by dissolving/dispersing Streptomycin to give an oil phase.

Preparation of Aqueous Phase: 90% Milli-Q water was taken in another manufacturing vessel and the aqueous phase was prepared by dissolving glycerol, drug followed by sodium hydrogen carbonate by stirring at 400 rpm on a magnetic stirrer maintaining temperature at 55° C. (50° C. to 60° C.).

Preparation of emulsion:

High Shear Homogenization: The oil phase containing drug was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 1-3 passes at different pressures varying from 10,000 psi to 18,000 psi followed by subsequent cooling the product to room temperature.

### Example 20

TABLE 41
Composition of OFA EE, Cannabidiol and THC emulsion

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | THC | 50 | 5.0 |
| 4 | Cannabidiol | 4 | 0.4 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) to give an oil phase. Dissolve THC into the oil mixture.

Preparation of Aqueous Phase: 90% Milli-Q water was taken in another manufacturing vessel and the aqueous phase was prepared by dissolving glycerol, followed by sodium hydrogen carbonate by stirring at 400 rpm on a magnetic stirrer maintaining temperature at 55° C. (50° C. to 60° C.).

Preparation of emulsion:

High Shear Homogenization: The oil phase containing drug was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse emulsion.

High Pressure Homogenization: The obtained coarse emulsion was subjected to high pressure homogenization for 1-3 passes at different pressures varying from 10,000 psi to 18,000 psi followed by subsequent cooling the product to room temperature.

### Example 21

TABLE 42
Composition of OFA EE, Cannabidiol and Pirfenidone Suspension

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Pirfenidone | 60 | 3.0 |
| 4 | Cannabidiol | 4 | 0.4 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) and followed by dispersing/dissolving Pirfenidone to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

Preparation of suspension:

High Shear Homogenization: The oil phase containing drug was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 1-3 passes at different pressures varying from 10,000 psi to 18,000 psi followed by subsequent cooling the product to room temperature.

### Example 22

TABLE 43
Composition of OFA EE, Cannabidiol and Nintedanib Suspension

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Nintedanib | 10 | 1.0 |
| 4 | Cannabidiol | 4 | 0.4 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) and followed by dispersing/dissolving Nintedanib to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

Preparation of suspension:

High Shear Homogenization: The oil phase containing drug was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 1-3 passes at different pressures varying from 10,000 psi to 18,000 psi followed by subsequent cooling the product to room temperature.

### Example 23

TABLE 44
Composition of OFA EE, Cannabidiol and Apremilast Suspension

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Apremilast | 10 | 1.0 |
| 4 | Cannabidiol | 4 | 0.4 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) and followed by dispersing/dissolving Apremilast to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

### Preparation of Suspension:

High Shear Homogenization: The oil phase containing drug was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 1-3 passes at different pressures varying from 10,000 psi to 18,000 psi followed by subsequent cooling the product to room temperature.

### Example 24

TABLE 45
Composition of OFA EE, Cannabidiol and Roflumilast Suspension

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Roflumilast | 3 | 0.3 |
| 4 | Cannabidiol | 4 | 0.4 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) and followed by dispersing/dissolving Roflumilast to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

### Preparation of Suspension:

High Shear Homogenization: The oil phase containing drug was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 1-3 passes at different pressures varying from 10,000 psi to 18,000 psi followed by subsequent cooling the product to room temperature.

### Example 25

TABLE 46
Composition of OFA EE, Cannabidiol and Isoniazid Suspension

| | | Qty Per mL | |
|---|---|---|---|
| S. No | Ingredients | (mg) | % (w/v) |
| 1 | Omega-3-Acid Ethyl Ester | 60 | 6.0 |
| 2 | Egg Lecithin (Lipoid E 80) | 10 | 1.0 |
| 3 | Isoniazid | 15 | 1.5 |
| 4 | Cannabidiol | 4 | 0.4 |
| 5 | Glycerol | 20 | 2.0 |
| 6 | Sodium Hydrogen Carbonate | 0.1 | 0.01 |
| 7 | Milli-Q Water | Q.S to 1 mL | Q.S to 100% |

### Manufacturing Procedure:

All the ingredients were weighed as per manufacturing formula.

Preparation of Oil Phase: Accurately weighed quantity of Omega 3 Acid Ethyl Ester was taken in a manufacturing vessel to which egg lecithin was added and dissolved by heating on a water bath at 55° C. (50° C. to 60° C.). Weighed quantity of Cannabidiol was added to the above mixture and dissolved by heating on a water bath at 45° C. (40° C. to 50° C.) and followed by dispersing/dissolving Isoniazid to give an oil phase.

Preparation of Aqueous Phase: 90% Milli-Q water was taken in another manufacturing vessel and the aqueous phase was prepared by dissolving glycerol followed by sodium hydrogen carbonate by stirring at 400 rpm on a magnetic stirrer maintaining temperature at 55° C. (50° C. to 60° C.).

### Preparation of Suspension:

High Shear Homogenization: The oil phase containing drug was added into the aqueous phase, under homogenization at 8500 rpm by maintaining the product temperature at 45° C. (40° C. to 50° C.) for 15 min. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 1-3 passes at different pressures varying from 10,000 psi to 18,000 psi followed by subsequent cooling the product to room temperature.

1. A novel inhalation formulation of omega 3 fatty acids, comprising therapeutically effective dose of docosahexaenoic acid (DHA), docosapentaenoic acid (DPA), or eicosapentaenoic acid (EPA) or a combination of any two or all the three of DHA, DPA, and EPA.

2. The formulation of claim 1, wherein the fatty acids are present as free fatty acids (FFA) or ethyl esters (EE) or phospholipids (PL) or non-phosphate-containing glycerolipids selected from the group comprising triacylglycerol, diacylglycerol, and/or monoacylglycerol.

3. The formulation of claim 1, wherein the formulation is suitable for nebulization or inhalation by any method known in the art.

4. The formulation of claim 1, wherein the formulation additionally comprises phospholipid.

5. The formulation of claim 1, wherein the formulation is a suspension or emulsion.

6. The formulation of claim 1, wherein the formulation additionally comprises a therapeutically active ingredient selected from the group comprising pirfenidone, apremilast, roflumilast, tiotropium bromide, nintedanib, isoniazid, streptomycin, montelukast, tetrahydrocannabinol and thereof.

7. The formulation of claim 1 used for treatment of COVID-19 and other lung disorders.

8. The formulation of claim 1 used for reducing pathological levels of IL-6 in the lung.

9. The formulation of claim 1 used for reducing pathological levels of TNF-alpha in the lung.

10. The formulation of claim 1 used for reducing pathological levels of IL-10 in the lung.

11. The formulation of claim 1 used for reducing pathological levels of TGF-beta in the lung.

12. The formulation of claim 1 used for treatment of disorders selected from the group comprising bronchiolitis, asthma, cystic fibrosis, COPD, pneumonia, tuberculosis, emphysema, pulmonary edema, lung cancer, acute respiratory distress syndrome (ARDS), asbestosis, bronchiectasis, interstitial lung disease (ILD) including sarcoidosis, idiopathic pulmonary fibrosis and CNS disorders.

13. The formulation of claim 1 further comprising a therapeutically effective dose of melatonin.

14. The formulation of claim 1 further comprising a therapeutically effective dose of budesonide.

15. The formulation of claim 1 further comprising a therapeutically effective dose of cannabidiol.

16. The formulation of claims 13, is administered to patients for whom NSAIDs are contraindicated.

17. The formulation of claims 13, is administered to patients to prevent or treat conditions selected from the group comprising acute pulmonary thrombosis, acute or chronic pulmonary inflammation, inflammatory conditions of the heart and its blood vessels or to alleviate bronchoconstriction optionally in combination with a fast acting bronchodilator.

18. A formulation comprising a therapeutically effective dose of phospholipid in which the phospholipid delivers docosahexaenoic acid (DHA), docosapentaenoic acid (DPA), or eicosapentaenoic acid (EPA) or any combination of two or all three of DHA, DPA, and EPA.

19. The formulation of claim 18, further comprising a therapeutically effective dose of melatonin.

20. The formulation of claim 18, further comprising a therapeutically effective dose of budesonide.

21. The formulation of claim 18, further comprising a therapeutically effective dose of cannabidiol.

22. A formulation containing a therapeutically effective dose of glycerolipid in which the glycerolipid delivers docosahexaenoic acid (DHA), docosapentaenoic acid (DPA), or eicosapentaenoic acid (EPA) or any combination of two or all three of DHA, DPA, and EPA.

23. The formulation of claim 22, further comprising a therapeutically effective dose of melatonin.

24. The formulation of claim 22, further comprising a therapeutically effective dose of budesonide.

25. The formulation of claim 22, further comprising a therapeutically effective dose of cannabidiol.

26. A novel inhalation formulation of omega 3 fatty acids, comprising therapeutically effective dose of docosahexaenoic acid (DHA), docosapentaenoic acid (DPA), or eicosapentaenoic acid (EPA) or a combination of any two or all the three of DHA, DPA, and EPA, wherein the formulation is suspension or emulsion.

27. The formulation of claim 14 is administered to patients for whom NSAIDs are contraindicated.

28. The formulation of claim 15 is administered to patients for whom NSAIDs are contraindicated.

29. The formulation of claim 14 is administered to patients to prevent or treat conditions selected from the group comprising acute pulmonary thrombosis, acute or chronic pulmonary inflammation, inflammatory conditions of the heart and its blood vessels or to alleviate bronchoconstriction optionally in combination with a fast acting bronchodilator.

30. The formulation of claim 15 is administered to patients to prevent or treat conditions selected from the group comprising acute pulmonary thrombosis, acute or chronic pulmonary inflammation, inflammatory conditions of the heart and its blood vessels or to alleviate bronchoconstriction optionally in combination with a fast acting bronchodilator.
